(19) United States
(12) Patent Application Publication
(10) Pub. No.: US 2025/0345571 A1
(43) Pub. Date: Nov. 13, 2025

(54) INFLATION DEVICE FOR INFLATING A BALLOON CATHETER

(71) Applicant: B. Braun Melsungen AG, Melsungen (DE)

(72) Inventors: Felix Fulda, Berlin (DE); Andreas Stolle, Berlin (DE)

(21) Appl. No.: 18/868,745

(22) PCT Filed: May 24, 2023
(86) PCT No.: PCT/EP2023/063973

§ 371 Date: Nov. 23, 2024

(30) Foreign Application Priority Data
May 25, 2022 (DE) 10 2022 113 211.0

(51) Int. Cl. A61M 25/10; A61M 39/22

(52) U.S. Cl. CPC A61M 25/10182; A61M 39/22; A61M 2205/3327; A61M 2205/3331; A61M 2205/3592; A61M 2205/50; A61M 2205/6072

(57) ABSTRACT

An inflation device for inflating a balloon catheter for angioplasty includes a syringe with a hollow syringe body, a syringe opening joined to the syringe body and connectable to the balloon catheter, and a syringe plunger arranged in the syringe body. The syringe plunger, together with a syringe-body wall, delimits a pressure chamber connected to the syringe opening and is axially displaceable in the syringe body to change a volume of the pressure chamber. The syringe is partially filled with a contrast medium mixture and has an identifier, preferably in the form of a machine-readable code, which contains data regarding an amount of contrast medium mixture contained in the pressure chamber and/or a mixing ratio of the contrast medium mixture.

### CROSS-REFERENCE TO RELATED APPLICATIONS

This application is the United States national stage entry of International Application No. PCT/EP2023/063973, filed on May 24, 2023, which claims priority to German Application No. 10 2022 113 211.0, filed on May 25, 2022. The contents of International Application No. PCT/EP2023/063973 and German Application No. 10 2022 113 211.0 are incorporated by reference herein in their entireties.

### FIELD

The present disclosure relates to an inflation device for inflating a balloon catheter for angioplasty or for inflating other medical devices based on the balloon catheter technology.

### BACKGROUND

Angioplasty is a treatment procedure for the (minimally invasive) widening of constricted or blocked blood vessels, where a balloon catheter is inserted into the blood vessel and the blood vessel is widened by expanding the balloon catheter, a procedure known as balloon dilation. Angioplasty is usually performed under X-ray control, where a contrast agent is injected into the blood vessel via the balloon catheter in order to detect a narrowed or blocked area of the blood vessel and the position of the balloon catheter in the blood vessel during the X-ray.

In the state of the art, a manual inflation syringe is used for angioplasty, with which, on the one hand, pressure of up to 30 bar may be built up by (manually) turning a syringe plunger of the inflation syringe, for example via a thread and a high transmission ratio, and on the other hand (without turning the syringe plunger) pressure may be released quickly, for example via unlocking by a ring or lever.

The manually set (excess) pressure in the inflation syringe leads to a corresponding inflation of the balloon catheter or a corresponding diameter of the balloon catheter. A correlation between the pressure set in the inflation syringe (and therefore in the balloon catheter) and the resulting diameter can be read off a diagram, usually in paper form, known as a compliance chart, so that a user may estimate and set the required pressure according to a target diameter. Sometimes the information on the relationship between the pressure in the balloon catheter and the diameter of the balloon catheter is also available in the form of a table, known as a compliance table. In this case, the required values may have to be interpolated from the specified values. In addition, the balloon catheter must be vented before inflation in order to exclude the presence of air in the balloon catheter during pressure build-up. To do this, the inflation syringe is connected to the balloon catheter and pulled up, creating a negative pressure in the inflation syringe and sucking the air out of the balloon catheter.

However, the disadvantage of manual adjustment is that, in particular during pressure build-up, it may lead to misapplication in several respects: For example, pressures above a permissible range may be generated due to a lack of pressure limitation options. Furthermore, reading errors may occur on a pressure gauge for (analog) pressure display or on the diagram, which in turn leads to incorrectly set pressures. Interpolation errors may also occur when using compliance tables.

It is also known from the state of the art to monitor by sensors how much fluid is supplied to the balloon catheter from the syringe. For example, the publication US 2007/0 213 656 A1 discloses a device for detecting and outputting data during the inflation of an expandable component, such as a balloon catheter, via fluid supply from a reservoir, such as a syringe chamber. The device has sensors with which the pressure, the flow rate and/or the volume of the fluid for expanding the component may be detected. The device also has an indicator that displays the pressure, flow rate and/or volume qualitatively or quantitatively.

Furthermore, in the prior art, the contrast agent is manually filled into the inflation syringe before angioplasty. For this purpose, the undiluted contrast agent is diluted until a desired mixing ratio is obtained for use in the inflation syringe, since the mixing ratio affects the radiopaque properties. The inflation syringe is then inflated with the diluted contrast agent until the required amount of contrast agent has been absorbed into the inflation syringe, which may vary depending on whether the procedure is an angioplasty (percutaneous transluminal angioplasty, PTA) in peripheral vessels or a coronary angioplasty (percutaneous transluminal coronary angioplasty, PTCA), for example. When the negative pressure is released, the vacuumed lumen of the balloon catheter fills with the contrast agent mixture contained in the inflation syringe. An excessive amount of contrast agent may have a disadvantageous effect on sufficient deflation of the balloon catheter before angioplasty or may lead to a prolongation of the deflation of the balloon catheter after angioplasty and the associated complication of removing the balloon catheter. Thus, when manually filling the inflation syringe with contrast agent, an incorrectly selected or incorrectly set amount of contrast agent may lead to incorrect use.

When a balloon catheter is inflated in the patient, this leads to a temporary blockage of the blood vessel. It is therefore essential to ensure that the inflation time is not too long, since this may lead to pain for the patient, who is fully conscious during such an intervention. Blocking the vessel for too long may also lead to necrosis. Undiluted contrast agent is much more viscous than water, so the contrast agent is usually diluted to the point where it still contrasts recognizably with the surrounding tissue in the X-ray image, but is as thin as possible. There are also very rare situations in which a balloon catheter gets stuck in the patient's vessel after deflation. In such a case, the doctor may decide to deliberately burst the balloon catheter in the vessel in order to remove it. In such a case, there must be no gas in the balloon catheter, since this would spontaneously expand explosively and could cause the vessel to burst. In addition, a high-dose contrast agent would be critical to the patient's health in this case.

### SUMMARY

Accordingly, it is the object of the present disclosure to avoid the disadvantages of the prior art and to provide an inflation device for inflating a balloon catheter for angioplasty, with which a pressure required for inflation can be built up and in which the risk of misapplication may be excluded or at least reduced.

Accordingly, the object of the present disclosure is solved by an inflation device for inflating a balloon catheter for angioplasty, comprising a syringe. The syringe has a hollow syringe body (syringe cylinder), a syringe opening connected to the syringe body and connectable to the balloon catheter (i.e. a balloon catheter lumen), and a syringe plunger arranged in the syringe body.

Together with a syringe-body wall, the syringe plunger delimits a pressurized room of the syringe connected to the syringe opening. This means that a syringe volume configured within the syringe-body wall is divided by the syringe plunger into the pressurized room and a residual room/empty space (residual volume/empty volume). The syringe plunger is axially displaceable in the syringe body in order to change the volume of the pressurized room. This means that the pressurized room varies in size depending on the axial position of the syringe plunger. This means that an overpressure may be generated by reducing the size of the pressurized room, i.e. liquid or air contained in the pressurized room in particular may be compressed or conveyed out of the syringe via the syringe opening. By enlarging the pressurized room, a negative pressure may be generated, i.e. liquid or air in particular contained in the pressurized room may be expanded or sucked into the syringe via the syringe opening.

According to the disclosure, the syringe is partially filled with a contrast agent mixture. This means that a (certain) amount of the contrast agent mixture, i.e. a contrast agent diluted in a (certain) mixing ratio, is contained in the pressurized room and the syringe is not completely filled, so that a certain empty volume is present. Accordingly, the syringe plunger is located in a central axial position (not in an end position), so that it may be displaced from this axial position in both axial directions, and thus both the contrast agent mixture may be compressed and/or may be conveyed out of the pressurized room and, in particular, air may be sucked into the pressurized room. The syringe is (already) partially filled/loaded in particular before inflation of the balloon catheter and/or in a sealed state of the syringe, i.e. not completely. Furthermore, the syringe according to the disclosure has a labeling which contains data about the quantity (/filling quantity) of the contrast agent mixture contained in the pressurized room and/or the mixing ratio of the contrast agent mixture. In particular, the labeling contains data of a filling quantity of the syringes partially filled before inflation of the balloon catheter and/or in the sealed state. This means that the labeling in particular indicates to what extent the syringe is initially (partially) filled. The labeling may preferably be configured in the form of a machine-readable code, for example a barcode, QR code or RFID transponder.

The core of the disclosure is therefore that the syringe is already partially filled, i.e. with a sufficiently large amount of contrast agent mixture to build up pressure in the balloon catheter and a sufficiently large residual draw volume, and is provided labeled for the user so that misapplication by the user can be ruled out when filling with an amount that is too large or too small or with an unsuitable mixing ratio or when selecting the partially filled syringe. The user is therefore not only relieved of the filling step of the syringe, in which the undiluted contrast agent has to be diluted to the correct mixing ratio and the correct quantity has to be drawn up with the syringe, but is also relieved of the identification of the same data, so that use is as error-free as possible. The use of a machine-readable code also makes it possible to check the selection or provide automated support.

In contrast to other syringe applications, such as drug delivery, with balloon catheter inflation it is not predominantly crucial that the liquid delivery via the syringe is precisely dosed and must be guaranteed at all times in order to avoid incorrect dosing or abrupt termination of the liquid delivery for patient safety. In contrast, when inflating balloon catheters, it is imperative that the amount of contrast agent mixture contained is sufficiently large, but also sufficiently small, in order to provide the necessary residual volume for venting on the one hand, and to be able to quickly empty the contrast agent mixture previously supplied to the balloon catheter on the other hand. The use of balloon catheters for inflating is therefore not comparable with other syringe applications in which the residual draw volume is not important and therefore the prefilled syringes are usually completely filled for cost reasons.

Iopromide, iodixanol, ioxaglate, iohexol, iopamidol, iomeprol, iomeron, gadodiamide or gadolinium may be used as a contrast agent, for example. For dilution, the contrast agent is preferably diluted with a saline solution, e.g. NaCl 0.9%. Gadodiamide or gadolinium may also be used undiluted. A ratio of contrast agent to diluent of 1:1 to 1:3, preferably about 1:2, is suitable as a mixing ratio.

According to a preferred embodiment, the syringe opening may have a sealed closure. This may prevent the case that the labeling data at the time when the closure is (still) sealed does not match the actual filling quantity in the syringe. This has the advantage that once the syringe has been filled with the contrast agent mixture and has been sealed, it is not possible to change the filling (in particular to remove part of the contrast agent mixture) without this being apparent. Consequently, accidental misapplication of a syringe that has already been partially emptied (beyond the initial filling quantity), for example, can be ruled out. In addition, contamination of the syringe or the filling can be prevented.

According to a preferred embodiment, the syringe may be configured as a single-use (one-time/disposable) product. This means that the syringe as such is not suitable for reprocessing and multiple use (for different patients), among other things for reasons of sterility and patient safety. However, the syringe may be used for the inflation of several balloon catheters one after the other, for example for balloon catheters of different sizes inserted one after the other.

According to a preferred embodiment, the syringe, in particular before inflation of the balloon catheter and/or in the sealed state of the syringe, may be filled with the contrast agent mixture to 10 to 80%, preferably to 15 to 70%, more preferably to 20 to 60%, particularly preferably to 25 to 50%, of a total volume of the syringe body. The syringe may have a syringe diameter of 5 to 50 millimeters, preferably 15 to 30 millimeters. Furthermore, the syringe may have a total filling volume, i.e. a maximum filling volume (with the syringe plunger fully extended/retracted), of 10 to 250 milliliters, preferably 20 to 100 milliliters. In addition, the amount of contrast agent mixture may be, for example, 5 to 50 milliliters, preferably 10 to 25 milliliters. These sizes and filling quantities have proven to be particularly suitable for the inflation of balloon catheters for angioplasty.

According to a preferred embodiment, the labeling may contain data of a syringe type of the syringe. For example, the syringe type data may be product-specific data that is relevant for the use of the syringe, such as a (total) filling volume, a batch number, an article number and/or a maximum permissible pressure. This may simplify the correct use of the syringe, reduce the risk of misapplication, and partially automate or automate the use of the inflation device. The syringe may have a single labeling (/identification codes), in which the data on the syringe type and the contrast agent mixture are contained together, or several (e.g. two) individual labelings (/identification codes), in which the data on the syringe type and the contrast agent mixture are contained separately from each other. For example, the separate labelings may also be readable in different ways.

According to a preferred embodiment, the inflation device may comprise a pressure sensor (/pressure plunger) for detecting the pressure in the pressurized room, in particular arranged in the syringe plunger. According to an alternative preferred embodiment, the pressurized room may be connectable with a pressure sensor (/a separate measuring instrument) for detecting the pressure in the pressurized room, in particular via a terminal connected to the syringe opening, preferably in the form of a three-way stopcock. This means that the pressure in the pressurized room may not only be displayed and read directly via a manual pressure gauge, but may also be measured and preferably processed automatically. This means that reading errors by a user may be avoided. In addition, a sterile filter may be arranged in particular between the (external) pressure sensor and the pressurized room in order to avoid contamination of the contrast agent mixture.

According to the preferred embodiment, the inflation device may have a control device to which the pressure sensor may be connected wirelessly, for example by radio, or by cable to transmit the detected pressure. This allows the pressure to be read out and preferably to be further processed for automatic control.

According to the preferred embodiment, the inflation device may have a pressure relief device for limiting a maximum pressure in the pressurized room. Preferably, the pressure relief device may be configured such that it prevents a reduction of the pressurized room as soon as the pressure in the pressurized room reaches the maximum permissible pressure of the syringe. For example, the pressure relief device may prevent an overpressure by inhibition, in particular when the pressure measured at the pressure sensor reaches the maximum permissible pressure that can be read from the labeling. In this way, incorrect use may preferably be prevented automatically/in an automated way.

According to a preferred embodiment, the inflation device may have a position sensor for detecting a position of the syringe plunger. This has the advantage that the filling level/degree of filling of the syringe (and thus the quantity of contrast agent mixture conveyed out of the pressurized room) may also be monitored. Furthermore, this may prevent the syringe plunger from moving to its end positions.

According to a preferred embodiment, the inflation device may have a drive unit that is mechanically coupled or coupleable to the syringe plunger in order to axially displace the syringe plunger to reduce the pressurized room and to enlarge the pressurized room. The drive unit may be configured and coupled to the syringe plunger in such a way that it realizes a force adjustment and a quick adjustment. During force adjustment, pressures of 6 to 30 bar may be applied to the syringe plunger. For example, the force may be adjusted by rotating the syringe plunger, which is converted into an axial displacement via a thread. A force adjustment is required in particular for inflating itself, i.e. for feeding the contrast agent mixture into the balloon catheter under high pressure. This means that in particular the syringe plunger may be advanced/the pressurized room may be reduced with high force/(significantly) increased force compared to the quick adjustment. With the quick adjustment, speeds of at least one syringe adjustment length/second may be achieved. For example, the quick adjustment may be made by loosening a thread engagement. A quick adjustment is required in particular for deflating the balloon catheter and for venting the balloon catheter. This means that, in particular, the syringe plunger may be retracted/the pressurized space may be enlarged at high speed/(significantly) increased speed compared to the force adjustment. In other words, the drive unit is preferably configured in such a way that it has a force-applying component and a component for rapid displacement.

According to a preferred embodiment, the inflation device may be connectable to a vent valve for venting the pressurized room, in particular via a terminal or the terminal connected to the syringe opening, preferably in the form of a three-way stopcock or the three-way stopcock. Thus, deflation of the balloon catheter may be achieved particularly simply by first establishing a connection between the syringe opening and the balloon catheter, in particular by opening a terminal of the three-way stopcock connected to the balloon catheter, and retracting the syringe plunger, whereby air contained in the balloon catheter is sucked out of the balloon catheter into the pressurized room, and then the connection between the syringe opening and the balloon catheter is interrupted, in particular by blocking the terminal of the three-way stopcock connected to the balloon catheter, a connection is established between the syringe opening and the vent valve, in particular by opening the terminal of the three-way stopcock connected to the vent valve, and the syringe plunger is advanced, whereby air contained in the pressurized room is released out of the pressurized room via the vent valve to the environment. This venting process may also be carried out several times until the balloon catheter is completely deflated. Inflation may then take place by feeding the contrast agent mixture into the balloon catheter.

Alternatively, the balloon catheter may be deflated by establishing a connection between the syringe opening and the balloon catheter and retracting the syringe plunger, whereby the air contained in the balloon catheter is sucked out of the balloon catheter into the pressurized room, wherein the air remains contained in the pressurized room during the subsequent inflation of the balloon catheter by supplying the contrast agent mixture. The air contained in the pressurized room is prevented from being fed in again by a position of the syringe in that the syringe is held with the syringe opening facing downwards during inflation, so that the air is only in an upper part of the pressurized room and only the contrast agent mixture located in a lower part of the pressurized room is fed to the balloon catheter.

Further alternatively, the balloon catheter may be deflated by establishing a connection between the syringe opening and the balloon catheter and retracting the syringe plunger, whereby the air contained in the balloon catheter is sucked out of the balloon catheter into the pressurized room, wherein the air sucked out of the balloon catheter into the pressurized room is discharged from the pressurized room via a semi-permeable membrane.

According to a further aspect of the disclosure, which may preferably be present in combination, but also independently of the partially filled and labeled syringe, the inflation device may comprise a balloon catheter connected or connectable to the syringe opening. The balloon catheter may have a labeling which contains data of a balloon catheter type of the balloon catheter. The labeling may preferably be configured in the form of a machine-readable code, for example a barcode, QR code or RFID transponder. For example, the balloon catheter type may be product-specific data that is relevant for the use of the balloon catheter, such as a deflation volume, a maximum inflation volume, a pressure-inflation diameter relation, an intended application or a mixing ratio of contrast agent mixture intended for the intended application, a batch number and/or an article number. This may simplify the correct use of the balloon catheter, reduce the risk of misapplication and automate or partially automate the use of the inflation device.

According to a preferred embodiment, the inflation device may be configured to recognize whether the syringe is suitable for complete inflation of the balloon catheter and/or for venting of the balloon catheter, depending on the type of balloon catheter and the quantity of contrast agent mixture or the syringe plunger position contained therein. Depending on the type of balloon catheter, a certain minimum amount of contrast agent mixture is required to be able to inflate the balloon catheter to the desired/specified target diameter. In addition, for venting the balloon catheter, depending on the type of balloon catheter, a certain minimum empty volume of the syringe is required in order to be able to suck the air out of the balloon catheter by pulling back the syringe plunger and, in particular, to be able to completely deflate the balloon catheter if pressurized room deflation is not possible. This makes it easy to recognize whether the selected, already partially filled syringe is suitable for the selected balloon catheter, thereby simplifying or partially automating/automating its use.

According to a preferred embodiment, the inflation device may be configured to recognize whether the syringe is suitable for the intended application of the balloon catheter, depending on the type of balloon catheter and the mixing ratio of the contrast agent mixture. In order to ensure suitable radiopaque properties, a specific mixing ratio of the contrast agent mixture is required depending on the type of balloon catheter. This makes it easy to recognize whether the radiopaque properties of the selected, already partially filled syringes are suitable for the selected balloon catheter, thereby simplifying or partially automating/automating their use.

In other words, the disclosure relates to an inflation device which is made up of two components, i.e. an electromechanical basic unit and a disposable product in the form of a syringe with a pressure body/piston. The basic unit has a mechanical drive via which the piston may be displaced within a (syringe) cylinder/body. The pressure generated may be determined by the force applied. The pressure may be controlled via the force on the piston. In addition, a position measurement may be provided to detect the position of the piston within the cylinder/body. This allows the filling level of the syringe to be monitored and prevents the piston from moving to its end positions. In addition, there may be a pressure sensor connected or connectable to the syringe, for example via an integrated three-way stopcock, with which the pressure in the syringe may be measured and/or the measured/measured pressure may be corrected. The syringe may have a product-specific QR code or barcode that may be scanned at the base unit so that a corresponding product is selected. In addition or alternatively, the corresponding product may be selected via operating elements of the basic unit. Furthermore, a valve may be connectable or connected to the syringe, for example via the integrated three-way stopcock, with which the lines may be vented or opened in the event of an emergency/fault, e.g. in the event of a power failure. The three-way stopcock may be located at the tip/opening of the one-way unit/syringe to allow the catheter and pressure measuring unit and valve to be connected to the basic unit.

Preferably, the one-way unit/syringe may be supplied already filled with contrast agent in order to ensure the same properties in terms of filling quantity and X-ray visibility, wherein the catheter is then filled and emptied according to constant rheological properties. The piston may be designed with a diameter of 5 to 50 mm, preferably 15 to 30 mm. A maximum stroke consists of a filling quantity of 10 to 250 ml, preferably 20 to 100 ml. A filling of the disposable product may be between 5 and 50 ml, preferably 10 to 25 ml, with contrast agent.

In order to deflate the one-way unit/syringe, the three-way stopcock is rotated such that the syringe volume is connected to the catheter lumen and the piston is pulled out (to the maximum). The piston is then quickly deflated, i.e. in less than 5 seconds, preferably in less than 2 seconds, so that the contrast agent is directed into the catheter lumen. After deflation, high pressures must be applied, for which an adequate design of the mechanism is necessary. In addition, rapid displacement is required, which is usually opposed by high forces.

Thus, the basic functions (and components) of the inflation device are the generation of a negative pressure for venting the catheter system, achieving and maintaining a (product-specific) positive pressure, setting a suitable pressure, enabling an emergency or rapid emptying of the catheter (emergency stop), achieving a minimum pressure of 6 atm, achieving a maximum pressure of 30 atm, providing a prefilled syringe with 10 to 30 ml contrast agent.

Alternatively, the pressure sensor and/or the valve in the base unit may be omitted and/or the three-way stopcock in the one-way unit may be omitted. The one-way unit or the basic unit may be configured to be pivotable and may be rotated manually or automatically to allow the one-way unit to be reused by venting the syringe plunger. A catheter must not be connected in this case.

### BRIEF DESCRIPTION OF THE DRAWINGS

FIG. 1 is a schematic representation of a basic principle of an inflation device according to the present disclosure;

FIG. 2 is a schematic representation of a cross-section of the inflation device from FIG. 1;

FIG. 3 is a schematic representation of a preferred embodiment of the inflation device according to the present disclosure; and

FIG. 4 is a schematic representation of a cross-section of the inflation device from FIG. 3.

### DETAILED DESCRIPTION

Embodiments of the present disclosure are described below based on the accompanying Figures.

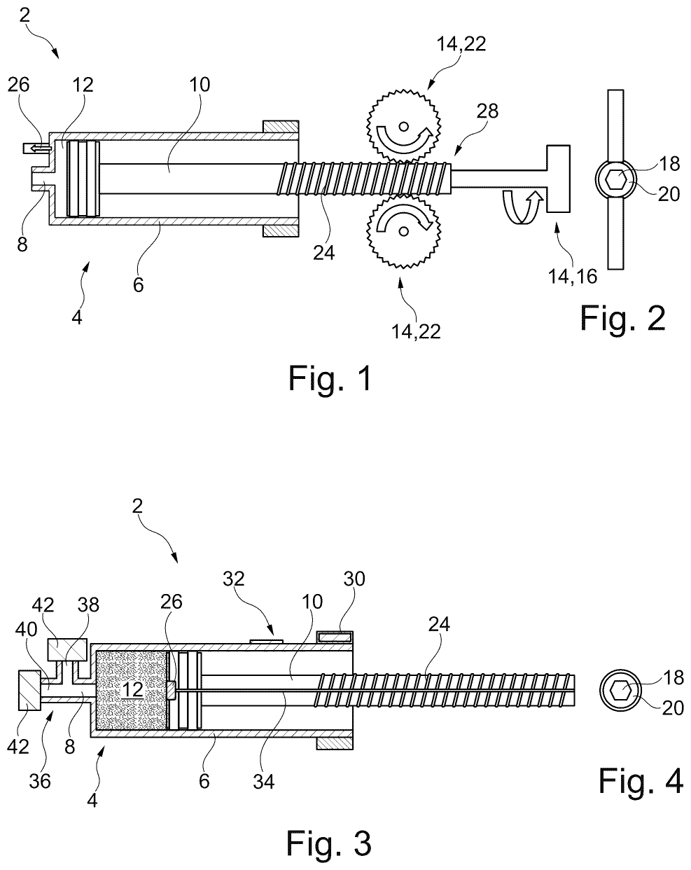

FIG. 1 shows a basic principle of an inflation device 2 according to the present disclosure. The inflation device 2 is used for inflating a balloon catheter (not shown) for angioplasty.

The inflation device 2 has a syringe 4. The syringe 4 has a hollow syringe body/syringe cylinder 6. In addition, the syringe 4 has a syringe opening 8 connected to the syringe body 6 and connectable to the balloon catheter. Furthermore, the syringe 4 has a syringe plunger 10 arranged in the syringe body 6. Together with a syringe-body wall, the syringe plunger 10 delimits a pressurized room 12 connected to the syringe opening 8. The pressurized room 12 is preferably sealed between the syringe-body wall and the syringe plunger 10 via a seal, in this case in the form of two seal rings. The syringe plunger 10 may be moved axially in the syringe body 6 to change the volume of the pressurized room 12 (syringe volume). Preferably, the syringe 4 may be configured as a disposable product.

The inflation device 2 has a drive unit 14. The drive unit 14 is mechanically coupled to the syringe plunger 10 in order to axially displace the syringe plunger 10. The drive unit 14 has a rotatable/rotationally drivable driveshaft 16, the rotation of which is coupled to a rotation of the syringe plunger 10 (about its longitudinal axis). In the embodiment shown, the driveshaft 16 has a form-fitting geometry 18, here in the form of an external hexagon, which engages in a corresponding mating form-fitting geometry 20 configured on the syringe plunger 10, here in the form of an internal hexagon (see FIG. 2), so that a torque may be transmitted form-fittingly from the driveshaft 16 to the syringe plunger 10.

In addition, the drive unit 14 has two gears 22 arranged opposite each other with respect to the syringe plunger 10. The gears 22 are in meshing engagement with an (external) thread 24 configured on the syringe plunger 10 (or may be brought into meshing engagement). The gears 22 are fixed with respect to the axial direction of the syringe plunger 10, so that the syringe plunger 10 is axially displaced, for example in the manner of a worm gear, when rotated about its longitudinal axis (by the driveshaft 16) and/or when the gears 22 are rotated about their longitudinal axis due to the toothing engagement with the gears 22.

This means that the syringe plunger 10 may be extended by turning the driveshaft 16 in one rotational direction (thus increasing the pressurized space 12) and may be retracted by turning the driveshaft 16 in the other rotational direction (thus reducing the pressurized space 12). The axial movement of the syringe plunger 10 may be accelerated by simultaneously rotating one or both gears 22 in the opposite direction to the force applied by the driveshaft 16, i.e. when at least one gear 22 is actively driven. When one or both gears 22 are at a standstill at the same time, i.e. when at least one gear 22 is braked, the syringe plunger 10 may be moved with high force. In addition, the syringe plunger 10 may be extended (and thus the pressurized space 12 may be enlarged) by rotating the gears 22 in one rotational direction and may be retracted (and thus the pressurized space 12 may be reduced) by rotating the gears 22 in the other rotational direction. The axial movement of the syringe plunger 10 may be accelerated by simultaneous rotation in the opposite direction to the force applied by the gears 22 or when the driveshaft 16 is stationary.

In addition, the inflation device 2 may have a sensor 26 connected to the pressurized room 12 for measuring a reaction force, via which a pressure in the pressurized room 12 may be measured or determined.

Furthermore, the inflation device 2 may have a position sensor 28, only indicated in FIG. 1, for detecting a position of the syringe plunger 10.

FIG. 3 shows a preferred embodiment of the inflation device 2 according to the present disclosure, the structure of which corresponds to the basic principle described with reference to FIG. 1. A representation of the drive device 14 is omitted in the schematic representation of FIG. 3.

The syringe 4 is partially filled with a contrast agent mixture. The contrast agent mixture is contained in the pressurized room 12. Preferably, the syringe may be filled to 10 to 80%, preferably to 15 to 70%, more preferably to 20 to 60%, particularly preferably to 25 to 50%, of a total volume of the syringe body 6 with the contrast agent mixture. This means that there is a residual volume/residual draw volume in which no contrast agent mixture is contained and the syringe is filled in the area of the pressurized room 12 and empty in the area of the residual volume. The syringe plunger 10 is therefore in a position between its end positions.

The syringe 4 has a labeling 30 which contains data on a quantity of the contrast agent mixture contained in the pressurized room 12 and/or a mixing ratio of the contrast agent mixture. In the embodiment shown, the labeling 30 is applied to an outer side of the syringe body 6. Preferably, the labeling 30 is configured in the form of a machine-readable code, for example a barcode, QR code or RFID transponder. In the embodiment shown, the labeling 30 is configured in the form of the RFID transponder, via which the amount of the contrast agent mixture and/or a mixing ratio of the contrast agent mixture may be clearly identified.

The syringe 4 has a further labeling 32, which contains data of a syringe type of the syringe. In the embodiment shown, the labeling 32 is applied to an outer side of the syringe body 6. Preferably, the labeling 32 is configured in the form of a machine-readable code, for example a barcode, QR code or RFID transponder. In the embodiment shown, the labeling 32 is configured in the form of a CR code. Data of the syringe type are, for example, a (total) filling volume of the syringe 2, a batch number, an article number and/or a maximum permissible pressure.

In addition, the syringe opening may have a sealed closure (not explicitly shown).

In the embodiment shown in FIG. 3, the pressure sensor 26 is configured as a pressure plunger, which is arranged on a side of the syringe plunger 10 facing the pressurized room. The pressure sensor 26 may be read out via a data line 34, for example by a control unit (not shown). Alternatively, the pressure sensor 26 may be read out wirelessly, for example via radio.

The syringe 4 has a three-way stopcock 26. A first terminal of the three-way stopcock 26 is connected to the syringe opening 8.

A second terminal 38 of the three-way stopcock 26 is connectable or connected to an (external) pressure sensor and/or a vent valve. A third terminal 40 of the three-way stopcock 26 is connectable or connected to the balloon catheter. The second terminal 38 and the third terminal 40 may, for example, be configured in the form of a Luer adapter/Luer lock 42.

In particular, the balloon catheter is deflated by first establishing a connection between the syringe opening 8 and the balloon catheter, in particular by opening the third terminal 40, and retracting the syringe plunger 10, preferably quickly. As a result, air contained in the balloon catheter is sucked out of the balloon catheter into the pressurized room 12. Then the connection between syringe opening 8 and balloon catheter is interrupted, in particular by blocking the third terminal 40, a connection between syringe opening 8 and vent valve is established, in particular by opening the second terminal 38, and the syringe plunger 10 is advanced. As a result, air contained in the pressurized room is released from the pressurized room 12 to the environment via the vent valve. This venting process may also be carried out several times until the balloon catheter is completely deflated.

In particular, the balloon catheter is inflated by establishing a connection between the syringe opening 8 and the balloon catheter (after deflating the balloon catheter), in particular by opening the third terminal 40, and advancing the syringe plunger 10, preferably with high force. Pressures of 6 to 30 bar may be built up and the balloon catheter may be expanded to a desired target diameter in accordance with a balloon catheter-specific pressure-inflation diameter ratio.

In particular, the balloon catheter is deflated by retracting the syringe plunger 10, preferably quickly (after the balloon catheter has been inflated and a blood vessel has been dilated as a result). As a result, the contrast agent mixture previously supplied to the balloon catheter is sucked out of the balloon catheter into the pressurized room 12 and the diameter of the balloon catheter is reduced so that it may be pulled out of the blood vessel.

It is possible that the syringe plunger 10 is initially withdrawn quickly in order to quickly relieve the patient of the pressure. After the quick relief, there may still be residual volume in the balloon catheter. The syringe plunger 10 may then be withdrawn further, for example slowly, in order to actively drain the residual volume/residual pressure in the balloon catheter and make the diameter of the deflated balloon catheter as small as possible so that it may be removed/pulled out of the patient as easily as possible.

1.-10. (canceled)

11. An inflation device for inflating a balloon catheter for angioplasty, the inflation device comprising:
a syringe having a syringe body that is hollow, a syringe opening connected to the syringe body and connectable to the balloon catheter, and a syringe plunger arranged in the syringe body, which together with a syringe-body wall delimits a pressurized room connected to the syringe opening and is axially displaceable in the syringe body in order to change a volume of the pressurized room,
the syringe being partially filled with a contrast agent mixture, and has a labeling which contains data about a quantity of the contrast agent mixture contained in the pressurized room.

12. The inflation device according to claim 11, wherein the syringe is partially filled with a contrast agent mixture before inflation of the balloon catheter and/or in a sealed state.

13. The inflation device according to claim 12, wherein the data comprises a filling quantity of the syringe partially filled before inflation of the balloon catheter and/or in the sealed state.

14. The inflation device according to claim 11, wherein the labeling comprises a machine-readable code.

15. The inflation device according to claim 14, wherein the machine-readable code is a barcode, QR code or RFID transponder.

16. The inflation device according to claim 11, wherein the data comprises a mixing ratio of the contrast agent mixture.

17. The inflation device according to claim 11, wherein the syringe opening has a sealed closure.

18. The inflation device according to claim 11, wherein the syringe is filled with the contrast agent mixture to 10 to 80% of a total volume of the syringe body.

19. The inflation device according to claim 11, wherein the labeling contains data of a syringe type of the syringe.

20. The inflation device according to claim 19, wherein the data comprises information about a filling volume, a batch number, an article number and/or a maximum permissible pressure.

21. The inflation device according to claim 11, further comprising a pressure sensor for detecting pressure in the pressurized room.

22. The inflation device according to claim 21, wherein the pressure sensor is arranged in the syringe plunger.

23. The inflation device according to claim 11, wherein the pressurized room is connectable with a pressure sensor for measuring pressure in the pressurized room.

24. The inflation device according to claim 23, wherein the pressurized room is connectable with the pressure sensor via a terminal connected to the syringe opening.

25. The inflation device according to claim 24, wherein the terminal comprises a three-way stopcock.

26. The inflation device according to claim 24, wherein the inflation device comprises at least one of:
a control device to which the pressure sensor is connectable wirelessly for transmitting a pressure measurement measured by the pressure sensor; and
a pressure relief device for limiting a maximum pressure in the pressurized room.

27. The inflation device according to claim 11, further comprising a position sensor for detecting a position of the syringe plunger.

28. The inflation device according to claim 11, further comprising a drive unit that is mechanically coupled or coupleable to the syringe plunger, in order to axially displace the syringe plunger to reduce the pressurized room and to enlarge the pressurized room, wherein the drive unit is configured and coupled to the syringe plunger in such a way that drive unit realizes a force adjustment for applying pressures of 6 to 30 bar.

29. The inflation device according to claim 11, wherein the inflation device is connectable to a vent valve for venting the pressurized room.

30. The inflation device according to claim 11, further comprising a balloon catheter connected or connectable to the syringe opening, the balloon catheter having a labeling containing data of a balloon catheter type of the balloon catheter.
